(19) United States
(12) Patent Application Publication
(10) Pub. No.: US 2004/0081686 A1
(43) Pub. Date: Apr. 29, 2004

(54) Use of particle vectors in immunomodulation

(75) Inventors: Roger Kravtzoff, Saint Felix De Lauragais (FR); Didier Betbeder, Aucamville (FR); Michel Major, Toulouse (FR); Olivier Balland, Pechabou (FR); Samir El Mir, Antony (FR); Frederic Triebel, Versailles (FR); Anne Casanova, Odars (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(73) Assignees: Biovector Therapeutics, Labege Cedex (FR); Institut Gustave-Roussy, Villejuif (FR)

(21) Appl. No.: 10/281,371

(22) Filed: Oct. 25, 2002

Related U.S. Application Data
(63) Continuation of application No. PCT/FR01/01289, filed on Apr. 26, 2001.

(30) Foreign Application Priority Data
Apr. 26, 2000 (FR) 00/05295
Apr. 26, 2000 (FR) 00/05304

(51) Int. Cl. A61K 48/00; A61K 38/19; A61K 38/20; A61K 9/127

(52) U.S. Cl. 424/450; 424/85.1; 424/85.2; 514/44

(57) ABSTRACT

The invention pertains to use of a vector of the type comprising a nonliquid hydrophilic core for the preparation of a medication intended for the treatment of cancers and/or viral diseases, the vector being combined in the medication with at least one substance other than an antigen capable of modulating the immune response.

The object of the present invention is a process for improving the immunomodulatory properties of a substance other than an antigen capable of modulating the immunologic response consisting of mixing said substance with hydrophilic particles possibly bearing ionic ligands and possibly covered by a layer of amphiphilic compounds. The invention concerns the treatment and/or prevention of diseases having an immunogenic nature such as cancers or infections.

The invention pertains more specifically to a therapeutic composition, notably a vaccine composition, and the process for its preparation.

The invention pertains more specifically to the use of particle vectors incorporating interleukin-2 for the preparation of medications intended for the treatment of cancers.

Cancer is a disease characterized by an uncontrolled proliferation of certain cells, which escape from the control of the immune system. In fact, the role of the immune system is not only to reject pathogenic foreign bodies (viruses, bacteria, parasites, etc.) which can penetrate into the human body, but also to eliminate cells presenting abnormal characteristics. It is sometimes possible that cancerous cells escape the vigilance of the immune system, for example by decreasing the expression of certain specific antigens of the tumor or of molecules involved in recognition by the immune system (proteins of the major histocompatibility complex). In other cases, although the tumors are strongly immunogenic, the immune system cannot combat them because the lymphocytes are in an anergic state in proximity to the cancerous mass.

A new strategy developed in recent years consists of stimulating the immune system by the injection of proteins of therapeutic value having a role in the regulation of the immune system. Among these proteins can be cited the cytokines of course, but also other agents such as the chemokines. Emphasis can be placed especially on the interleukins, the interferons, the TNF (tumor necrotizing factors), the TGF (transforming growth factors), the hemopoietic growth factors such as M-CSF and GM-CSF, as well as immune cell attraction factors such as MIF and RANTES.

Interleukin-2 (IL-2) is a cytokine of particular interest in the stimulation of the immune system. It is synthesized primarily by the T helper 1 lymphocytes (Th1) in response to stimulation by the antigene presented by the appropriate cells. It is involved in the development of specific and nonspecific immune responses; it interacts with numerous cells (B, T lymphocytes, natural killer cells NK) so as to activate them and induce their differentiation and/or their proliferation. Thus, it has been shown that IL-2 has antitumor properties; it can induce a regression of different solid tumors, melanomas, leukemias or lymphomas, etc.

However, the use of IL-2 in chemotherapy is beset by numerous problems. In order to obtain the desired effect, it is often necessary to administer high doses of protein which can induce disturbing side effects (fever, erythema, edema). Moreover, the purification of this protein is complicated; it is generally produced by genetic engineering and is therefore expensive. Moreover, cytokines are often unstable in solution even at 4° C., which creates a storage problem. Furthermore, the processes for the preparation of solutions containing IL-2 include a step involving addition of stabilizing protein, often albumin, which creates an issue for the regulatory agencies due to the risks created by the use of albumin.

The present invention proposes to resolve these various problems by implementing a new approach to formulating compositions containing IL-2 which makes it possible to obtain solutions that have higher activities than the usual formulations, especially with regard to antitumor activity. This new formulation also increases the stability of the IL-2. Moreover, the invention can be used to manufacture a medication that can be administered via the intranasal or oral route, which comprises an active principle delivery system exhibiting very considerable advantages over the previously described delivery systems. Furthermore, a formulation according to the invention allows elimination of albumin from compositions containing IL-2 intended for administration in injectable form.

The applicant developed its expertise in the preparation of synthetic particle vectors designated below as “BVSM™”.

A first type of BVSM and the process for its preparation were described in European patent no. 344 040. The particles comprise from the interior to the exterior:

a central nonliquid hydrophilic core constituted by a matrix of naturally or chemically cross-linked polysaccharides or oligosaccharides that can be modified by various ionic groups;

a layer of fatty acids grafted by covalent bonds to the core;

one or more lipid layers constituted especially by phospholipids.

The development of this first generation of BVSM led the applicant to conceive and prepare new BVSM with improved properties especially in relation to the transported active principles.

The patent applications published as WO 94/20078, WO 96/06638 and EP 782 851 describe these BVSM, their manufacture, their combination with various active principles and their use for the preparation of pharmaceutical compositions.

Thus, the hydrophilic core can be obtained by various previously described methods (such as in EP 344 040, WO 92/21329, WO 94/20078) and the cross-linking processes are known by the expert in the field; the polysaccharide can be positively or negatively charged by the grafting of ionic, cationic or anionic groups to the sugars composing the polymer. These groups can be selected from among the quaternary ammonium or carboxymethyl functional groups. The processes were described in WO 92/21329 and the previously cited patents. The charge of the polysaccharide core of the vectors of the compositions according to the invention is generally preferably a positive charge. Nevertheless, vectors with cores containing negative charges can sometimes be preferred, in particular in a composition for use in injectable form.

The previously cited patents also describe protocols that the expert in the field can use for the incorporation of the external lipid layer. This layer is preferably a DPPC-like compound, i.e., DPPC (dipalmitoyl phosphatidyl choline) or any other compound exhibiting the same properties as this product, to which cholesterol is added. However, other lipid compounds can be used, in particular phospholipids or ceramides, to which other constituents can be added, e.g., constituents of the biological membranes. The DPPC/cholesterol mass ratio is preferably 70/30.

The applicant's current know-how regarding the preparation of BVSM provides access to an extensive range of types of BVSM. These BVSM are formed of a cross-linked hydrophilic polymer, preferentially polysaccharides, in the form of nanoparticle gel; these BVSM are referred to as PSC (or NPS in French).

As stated above, this polymer can possibly bear positive or negative ionic ligands, i.e., BVSM referred to as positive or negative, respectively. This charged or uncharged polymer is optionally covered by a layer of amphiphilic compounds, preferentially phospholipids, i.e., light type BVSM described in PCT patent application WO 94/20078.

The size of the BVSM is comprised between 20 and 200 nm, preferentially between 50 and 100 nm, and more preferentially between 60 and 80 nm.

The BVSM can be sterilized by filtration and their combination with the active principles is implemented on the final manufactured BVSM product (Major et al., Biochem. & Biophys. Acta (1997), 1327, 3240). This makes it possible to work with particularly fragile biological molecules under optimal conditions.

The BVSM protect the biological molecules from degradation (Prieur R. et al. Vaccins (1996) Vol 14, No 6, pp 511-520, Combination of hCMV recombinant IE1 protein with 80 nm cationic biovectors: protection from proteolysis and potentiation of presentation to CD4).

It has also been shown that they augment the biological activity of peptides and proteins such as enzymes, antigens, etc.

The BVSM are known to be used as transporters of active substances, e.g., peptides, antigens or oligonucleotides. In this use there is formation of a real complex between the BVSM and the active substance, as in the ionic interactions between the cationic core of the BVSM and the anionic oligonucleotide. Thus there is formation of a stable ionic conjugate in a biological medium. In this type of preparation, the oligonucleotide/BVSM ratio is 5 to 10%, and the measured combination yield is greater than 90%.

The PCT patent application published as number WO 96/06638 discloses the augmentation of the immunogenicity of an antigen obtained by a simple antigen/BVSM mixture. In this case, the antigen is combined with the particle vector by ionic and/or hydrophobic bonds. In order to obtain this result, we incubated, e.g., 1 mg of protein GST-e4 per 10 mg of BVSM, which produced mean combination levels of 90% irrespective of the type of BVSM used (Prieur R. et al. Vaccins (1996) Vol 14, No 6, pp 511-520).

The applicant has now discovered that the BVSM make it possible to improve the immunomodulatory properties of substances other than an antigen capable of modulating the immunologic response. These substances more particularly are:

adjuvants capable of amplifying, regulating or modifying the immune response;

cytokines and chemokines the intrinsic property of which is to modify the activity of cells of the immune system;

immunosuppressants that are used in the treatment of allergies and/or transplant rejection;

substances capable of modifying the Th1/Th2 balance.

It should be kept in mind that the immune response is relayed by the lymphocyte cells, notably the B cells and the T lymphocytes. The T lymphocytes can be classified into two subtypes on the basis of their expression of CD4 and CD8 surface antigens. The CD4+cells are generally involved in the helper functions. In particular they secrete cytokines that induce the proliferation and the maturation of the lymphocyte cells. Thus, two profiles of T helper lymphocytes can be defined:

on the one hand, a Th1 profile which characterizes a cellular mediation response with induction of cytokines, notably of IL-2, IL-7 and interferon gamma, the stimulation of CTL and the induction of type IgG2a antibodies, and

on the other hand, a Th2 profile which characterizes a humoral mediation response, with induction of cytokines notably of IL-4, IL-5 and IL-10, and the presences of IgG1 and IgE type antibodies.

The protein antigens, whose presentation by the antigen presenting cells (APC) is primarily made by class II CMH, induce a response oriented toward the Th2. In contrast, the DNA which makes it possible after transfection to present the antigens directly on the class I CMH orients the immune response towards the Th1.

Certain adjuvants are known to orient the response primarily towards Th1 or Th2. For example, the aluminum salts which induce an IgG1 type serum response are considered to be Th2. In contrast, the adjuvants such as the derivative of lipid A (MPL) or the derivatives of QuilA, which induce an IgG2a and CTL response, are considered to be Th1. Thus, it would be useful to have available means that make it possible to act on the Th1/Th2 balance, which is precisely that which is proposed in the present invention.

The research performed by the applicant in the framework of the present invention shows that the BVSM does not act as an adjuvant because alone it does not induce activation of the immune system, but rather as a carrier enabling better penetration and/or better presentation of the antigen to the presenting cells. Thus, the results obtained showed the contribution of BVSM to the adjuvant power of CTB, MPL and ODN. Surprisingly, the adjuvant power is visualized in a different manner:

on the serum IgG response for which we obtained a strong synergy in the case of CTB or an addition of the responses in the case of the ODN CpG,

on the IgA response in which a synergy was clearly demonstrated for the MPL,

on the Th1/Th2 balance in which even in the case of a quantitatively equivalent response (IL-2), we observed a qualitative difference in the response notably in the IgG1/IgG2a balance which reflects a differential in the cell response.

Thus the invention first of all has as its object the use of a vector of the type comprising a nonliquid hydrophilic core for the preparation of a medication intended for the treatment of cancers and/or viral diseases, said vector being combined in the medication with at least one substance other than an antigen capable of modulating the immune response.

The vector is advantageously of the type comprising a nonliquid hydrophilic core and an external layer constituted at least in part of amphiphilic compounds associated with the core by hydrophobic interactions and/or ionic bonds. The external layer is formed by dipalmitoyl phosphatidyl choline (DPPC) and cholesterol.

The nonliquid hydrophilic core is constituted by a matrix of naturally or chemically cross-linked polysaccharides or oligosaccharides on which are grafted ionic ligands.

The ionic ligands preferably are ligands with a positive charge, such as the quaternary ammoniums.

Particle vectors with a nonliquid hydrophilic core and an external layer constituted of amphiphilic compounds have already been described, particularly in patent application WO 94/20078.

The hydrophilic core can be obtained by various previously described methods (such as in patents EP 344,040, WO 92/21329, WO 94/20078) and the cross-linking processes are known by the expert in the field; the polysaccharide can be charged positively or negatively by grafting ionic, cationic or anionic groups to the sugars composing the polymer. These groups can be selected from among the quaternary ammonium or carboxymethyl functional groups. The processes were described in WO 92/21329 and the previously cited patents. The charge of the polysaccharide core of the vectors of the compositions according to the invention is generally preferably a positive charge. Nevertheless, vectors with cores containing negative charges can sometimes be preferred, in particular in a composition for use in injectable form.

The previously cited patents also describe protocols that the expert in the field can use for the incorporation of the external lipid layer. This layer is preferably a DPPC-like compound, i.e., DPPC (dipalmitoyl phosphatidyl choline) or any other compound exhibiting the same properties as this product, to which cholesterol is added. However, other lipid compounds can be used, in particular phospholipids or ceramides, to which other constituents can be added, e.g., constituents of the biological membranes. The DPPC/cholesterol mass ratio is preferably 70/30.

In the use of the invention, the substance/particles weight ratio is comprised between circa 1% and 20%, preferably between circa 5% and 10%. The (substance)/(core+external layer) weight ratio is preferably 1 to 10.

A first class of substances other than an antigen capable of modulating the immune response comprises proteins of therapeutic value which play a role in the functioning of the immune system. More particularly, this substance is a cytokine or a chemokine, preferably selected from among the group comprising the interleukins, the interferons, the TNF, the TGF, G-CSF, CM-CSF, MIF, RANTES or a mixture of these substances.

A second class of substances other than an antigen capable of modulating the immune response, partially overlapping the preceding class, comprises the adjuvants. An “adjuvant” is understood to mean a molecule enabling amplification, regulation or orientation of the specific immune response of an antigen. Therefore, in order to evaluate the properties of the BVSM on the immunomodulatory power of substances other than antigens, various molecules belonging to the principal categories of adjuvants were tested in the framework of the present invention, such as bacterial products (endotoxins, wall components), cytokines and oligonucleotides (CpG). Among these, we can cite more particularly:

bacterial enterotoxins such as CT, CTB, LT,

liposaccharides derivatives such as MPL and derivatives of lipid A,

type QS21 derivatives of saponin,

ammonium salts and their derivatives, such as alum,

type DT or TT proteins,

or a mixture of these.

The substance other than an antigen capable of modulating the immune response is preferably selected from among:

adjuvants capable of amplifying, regulating or modifying the immune response;

cytokines or chemokines whose intrinsic property is to modify the activity of the cells of the immune system;

immunosuppressants that are used in the treatment of allergies and/or the rejection of transplants;

substances capable of modifying the Th1/Th2 balance.

Among these, the invention envisages more particularly as substance whose immunomodulatory properties one desires to improve, the cytokines that stimulate the activity of the immune cells, and among them, IL-2, IL-11 and GM-CSF. In fact, the cytokines stimulate or inhibit the activity of the immune cells.

One especially preferred form of implementing the invention concerns the use of a vector comprising:

a) a nonliquid hydrophilic core constituted by a naturally or chemically cross-linked matrix of polysaccharides or oligosaccharides on which are grafted ionic ligands,

b) an external layer constituted at least in part by amphiphilic compounds associated in the core by hydrophobic interactions and/or ionic bonds, and

c) incorporating interleukin-2

for the preparation of a medication intended for the treatment of cancers.

Such a pharmaceutical composition containing a vector comprising

a) a nonliquid hydrophilic core constituted by a naturally or chemically cross-linked matrix of polysaccharides or oligosaccharides on which are grafted ionic ligands,

b) an external layer constituted at least in part by amphiphilic compounds associated in the core by hydrophobic interactions and/or ionic bonds, and

c) incorporating interleukin-2 is also part of the invention.

In this use, the invention concerns the treatment of cancers of the nonimmunogenic or weakly immunogenic type and/or the treatment notably of viral infections such as AIDS or chronic hepatitis in which immunomodulators have been proposed for activating the immune response to complement the therapeutic treatments. A composition for the implementation of this use comprises hydrophilic particles, possibly bearing ionic ligands and possibly covered by a layer of amphiphilic compounds, in which are incorporated the proteins of therapeutic value or the adjuvants as defined above. Such a composition does not contain antigens.

The cancers that can be treated by a composition according to the invention can be of all types. In particular, we can cite the cancers of the ORL domain, of the esophagus, stomach, colon, rectum, prostate, liver, pancreas, breast, uterine neck or body, ovary, kidney, bladder, bone or thyroid. We can also add bronchopulmonary cancers, malignant melanomas, brain tumors, lymphomas (Hodgkins or non-Hodgkins), leukemias (myeloid or lymphoid) and cancers of unknown primary localization. These cancers can be primary or metastatic. They can be of the sarcoma, adenoma, carcinoma, adenocarcinoma, lymphoma, myeloma, glioma, blastoma or glioblastoma type. The cancers that can be treated by a composition according to the invention can be immunogenic or nonimmunogenic. The applicant has shown that the compositions according to the invention are particularly effective in the treatment and control of cancers that are weakly immunogenic or nonimmunogenic.

The compositions and medications according to the invention can be administered in various manners, in particular by the parenteral route. The compositions according to the invention can thus be administrated via the intravenous, subcutaneous, intramuscular or intradermal route. The administration can also be performed (and this is a great advantage of the invention) via the oral or intranasal route. The compositions enabling oral administration can be tablets, gels, powders, granules or oral suspensions or solutions. They also comprise the sublingual and buccal forms of administration.

Certain excipients can possibly be added to the pharmaceutical compositions according to the invention. Thus, it is possible to use all types of binding, surface-active, coating, dispersion or wetting agents.

The applicant demonstrated that these vectors are capable of fixing IL-2 in a very effective manner, that the protein conserves its biological activity and that unexpectedly this activity is augmented in relation to the unformulated protein.

The applicant also showed that a formulation according to the invention makes it possible to stabilize the protein in solution in the same manner as in the presence of albumin. Thus, the use of a vector comprising:

a) a nonliquid hydrophilic core constituted by a naturally or chemically cross-linked matrix of polysaccharides or oligosaccharides on which are grafted ionic ligands of positive or negative charge,

b) an external layer constituted at least in part by amphiphilic compounds associated in the core by hydrophobic interactions and/or ionic bonds, and

c) incorporating interleukin-2

for the preparation of a medication intended for the administration of said IL-2 protein in injectable form in the absence of albumin is also part of the invention.

Moreover, the applicant demonstrated that the vector-protein combination remains stable (measured by maintenance of the activity after multiple months of storage), which represents an improvement in relation to the prior technique in which therapeutic preparations of IL-2 could not be stored, resulting in increased cost.

Finally, the applicant demonstrated that surprisingly IL-2 formulated according to the invention and administered via the intranasal route has an activity that is similar to or greater than IL-2 administered via more conventional routes such as the subcutaneous route.

The use of a vector according to the invention thus makes it possible to overcome all of the various problems previously posed by the use of IL-2.

A vector according to the invention can therefore be used for the treatment of cancers when the goal is a potentiation of the immune response.

The invention also has as its object a process for improving the immunomodulatory properties of a substance other than an antigen capable of modulating the immune response, characterized in that it comprises mixing said substance with vectors of the type comprising a nonliquid hydrophilic core. The vectors are advantageously of the type comprising a nonliquid hydrophilic core and an external layer constituted at least in part of amphiphilic compounds associated with the core by hydrophobic interactions and/or ionic bonds. As previously stated, the nonliquid hydrophilic core is constituted by a matrix of naturally or chemically cross-linked polysaccharides or oligosaccharides on which are grafted ionic ligands, notably of positive charge, such as the quaternary ammoniums. The external layer is formed, e.g., of dipalmitoyl phosphatidyl choline (DPPC) and cholesterol, notably such that the DPPC/cholesterol mass ratio is 70/30. The substance/vectors weight ratio in the mixture is preferably comprised between circa 1% and 20%, preferably between circa 5% and 10%.

As previously stated, the substance other than an antigen capable of modulating the immune response is selected from among:

a protein of therapeutic value that plays a role in the functioning of the immune system, such as a cytokine or chemokine, preferably selected from among the group comprising the interleukins, the interferons, the TNF, the TGF, G-CSF, MIF, RANTES or a mixture of these;

an adjuvant, notably selected from among the group comprising the bacterial enterotoxins, the derivatives of liposaccharides, the oligonucleotides, saponins, ammonium salts and their derivatives, proteins of type DT or TT or a mixture of these;

a substance capable of modifying the Th1/Th2 balance;

an immunosuppressant.

The invention also pertains to a pharmaceutical composition, characterized in that it comprises:

a vector of the type comprising a nonliquid hydrophilic core constituted by a matrix of naturally or chemically cross-linked polysaccharides or oligosaccharides on which are grafted ionic ligands and an external layer constituted at least in part by amphiphilic compounds, associated with the core by hydrophobic interactions and/or ionic bonds, and

at least one protein of therapeutic value and/or an adjuvant or a mixture of these.

The vector, the protein of therapeutic value and the adjuvant being defined as previously stated.

The invention also pertains especially particularly to the use of vectors as defined above for the preparation of a therapeutic or prophylactic vaccine preparation, said particles being mixed in the composition with at least one antigen and at least one substance capable of modulating the immunologic response to said antigen. In the case of therapeutic use, the invention pertains most particularly to the treatment and/or prevention of viral diseases, notably AIDS and chronic hepatitis, but also cancers of an immunogenic nature.

Thus, the invention pertains most especially to a vaccine composition characterized in that it comprises vectors as previously defined and an antigen or a mixture of antigens and at least one substance capable of modulating the immunologic response to said antigen.

The substance capable of modulating the immunologic response to the antigen present in the composition of the invention is preferably an adjuvant. Among these adjuvants, the invention envisages bacterial products (endotoxins, wall components), cytokines and oligonucleotides (CpG). Among these, we can cite more particularly:

bacterial enterotoxins such as CT, CTB, LT,

liposaccharides derivatives such as MPL and derivatives of lipid A,

type QS21 saponin derivatives,

ammonium salts and their derivatives such as alum,

type DT or TT proteins,

or a mixture of these.

As previously stated, the substance/particles weight ratio is comprised between circa 1% and 20%, preferably between circa 5% and 10%.

The invention is most particularly suitable for the preparation of compositions for mucosal administration, notably nasal administration, but all other modes of administration, e.g., parenteral, can be envisaged.

Other characteristics and advantages of the invention will become apparent from the examples below concerning:

Example 1: the preparation and biological activity of BVSM-IL2.

Example 2: (ii) the effect in the mouse of BVSM on the immunogenicity of a trivalent split influenza vaccine with different adjuvants, and (iii) the effect in the mouse of the co-administration of the formulation and different adjuvants on the immunogenicity of a trivalent split influenza vaccine.

The examples below are intended to illustrate the invention without being limitative. In particular the values presented are given as examples and could be optimized upon implementation of the present invention.

Reference will be made in example 1 to the following figures:

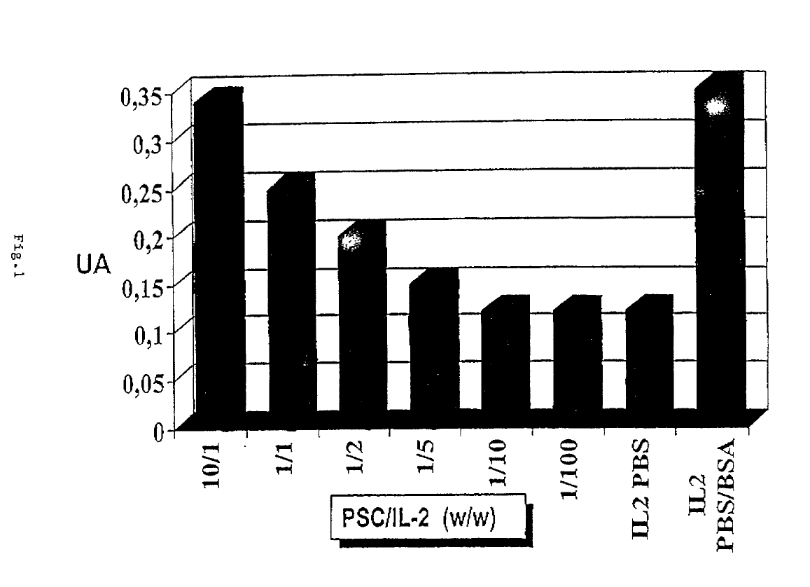

FIG. 1: ELISA analysis of the BVSM-IL2 interactions enabling, in particular, calculation of the optimal mass ration between the protein of interest and the vector. The values are given in Arbitrary Units (AU).

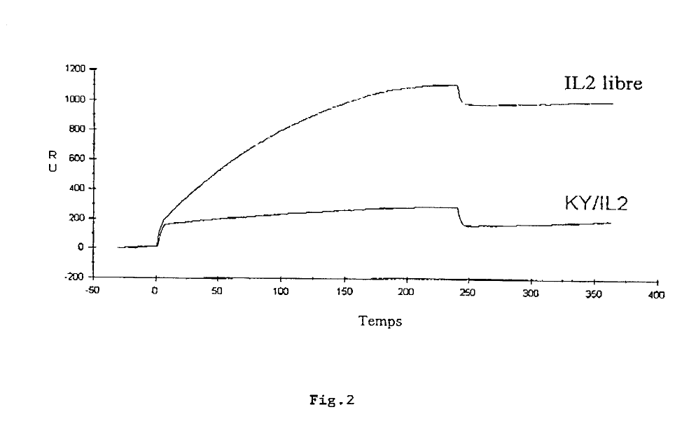

FIG. 2: Analysis of the BVSM-IL2 on a Biacore® device. Comparison of the refraction between a free IL-2 composition and the BVSM-IL2 composition. The values are given in Refraction Units (RU).

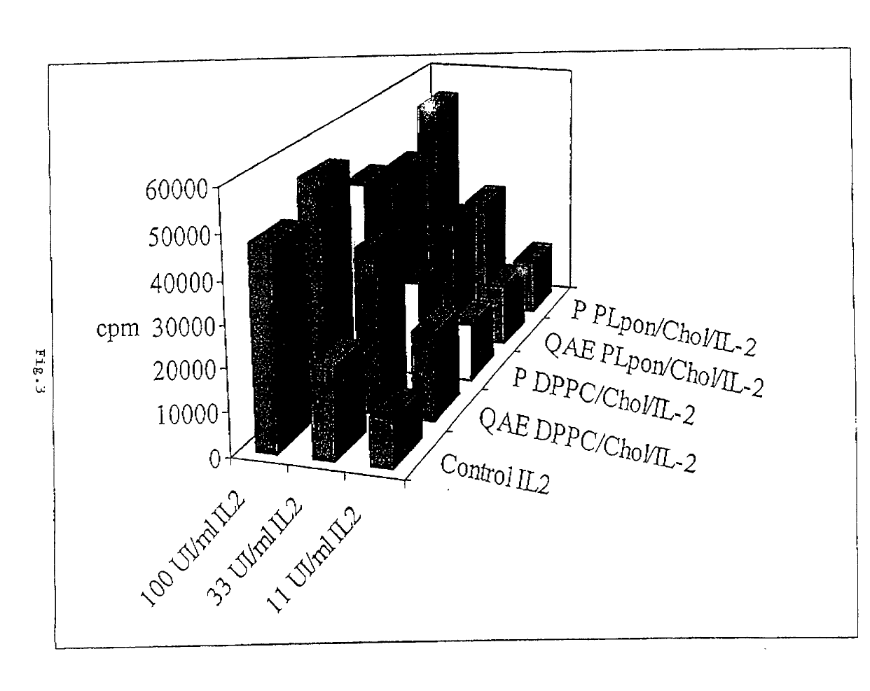

FIG. 3: Proliferation of stimulated peripheral mononuclear cells previously stimulated by PHA and by BVSM-IL2 of various compositions. The proliferation is measured by incorporation of H³-tagged thymidine and is proportional to the number of counts per minute (cpm) detected.

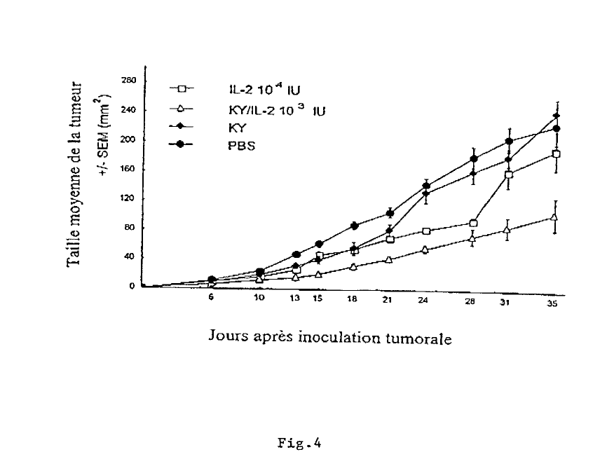

FIG. 4: Effect of different IL-2 formulations on the implantation and growth of a tumor in a TS/A model with co-administration. The symbols represent: PBS, saline buffer; IL-2, unformulated interleukin-2; KY, vector with cation core and DPPC/cholesterol layer; KY/IL-2, KY vector formulated with IL-2.

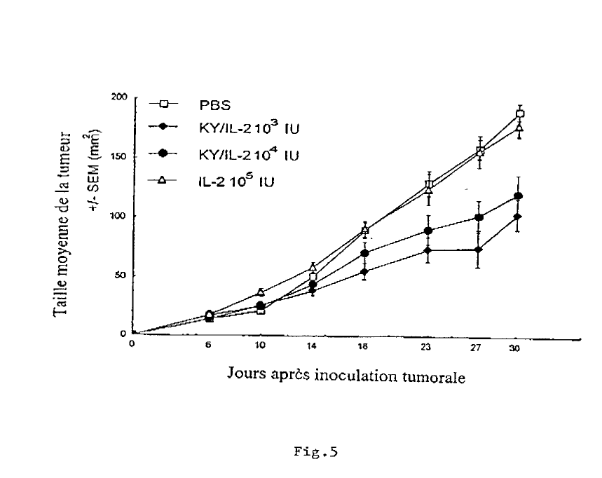

FIG. 5: Effect of different IL-2 formulations on the implantation and growth of a tumor in a TS/A model after administration in the contralateral flank of BALB/c mice. The symbols are the same as in the preceding figure; the numeric values indicated correspond to the IU values of IL-2.

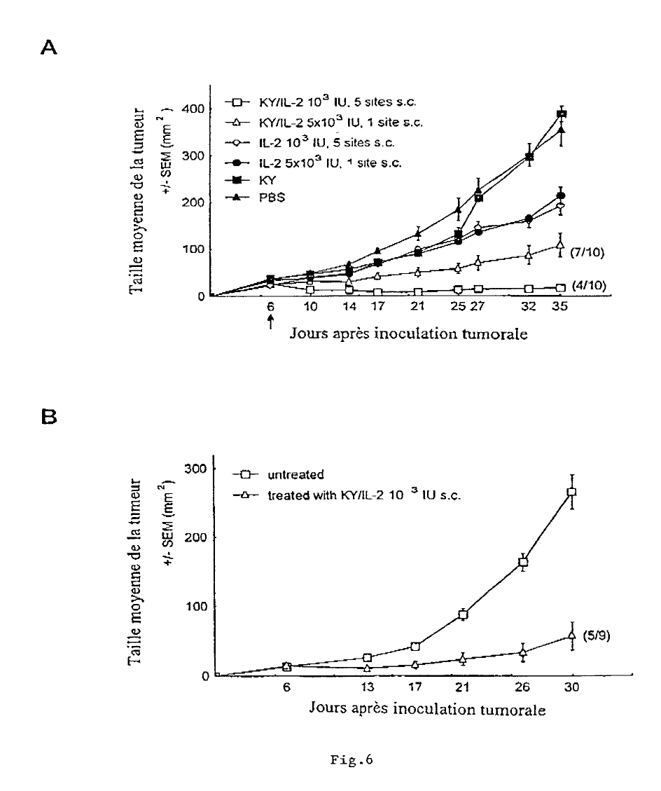

FIG. 6: A. Rejection properties of a TS/A tumor implanted by subcutaneous administration of KY/IL-2 in the contralateral flank of BALB/c mice at one or five site(s), six days after implantation. The symbols are the same as in the preceding figure; the numeric values indicated correspond to the IU values of IL-2. B. Protection of cured mice after re-injection of TS/A tumor cells. The number of mice presenting tumors is indicated between parentheses in both figures.

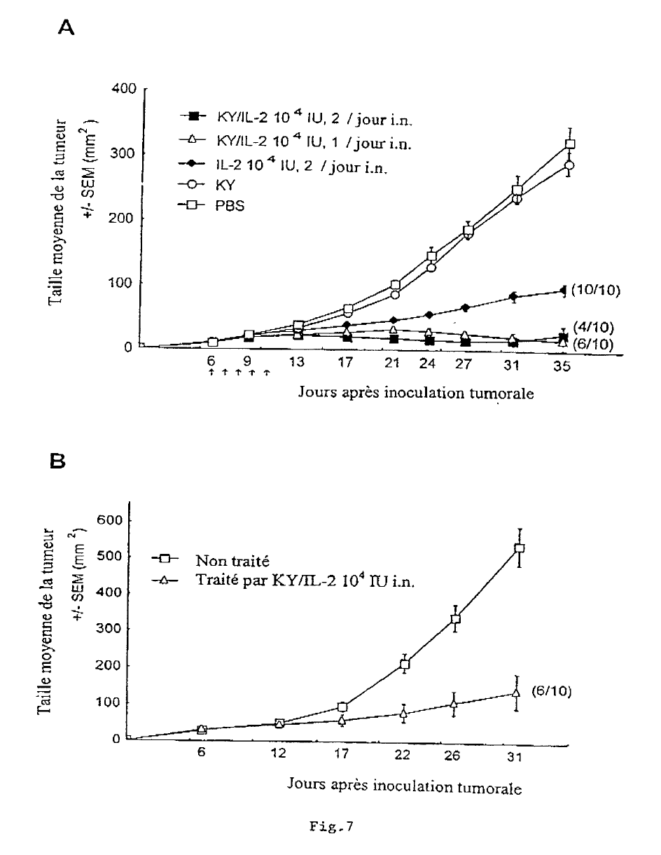

FIG. 7: A. Rejection properties of a TS/A tumor implanted by intranasal administration of KY/IL-2 in the contralateral flank of BALB/c mice once or twice per day for five days, six days after the implementation. The symbols are the same as in the preceding figure; the numeric values indicated correspond to the IU values of IL-2. B. Protection of cured mice after re-injection of TS/A tumor cells. The number of mice presenting tumors is indicated between parentheses in both figures.

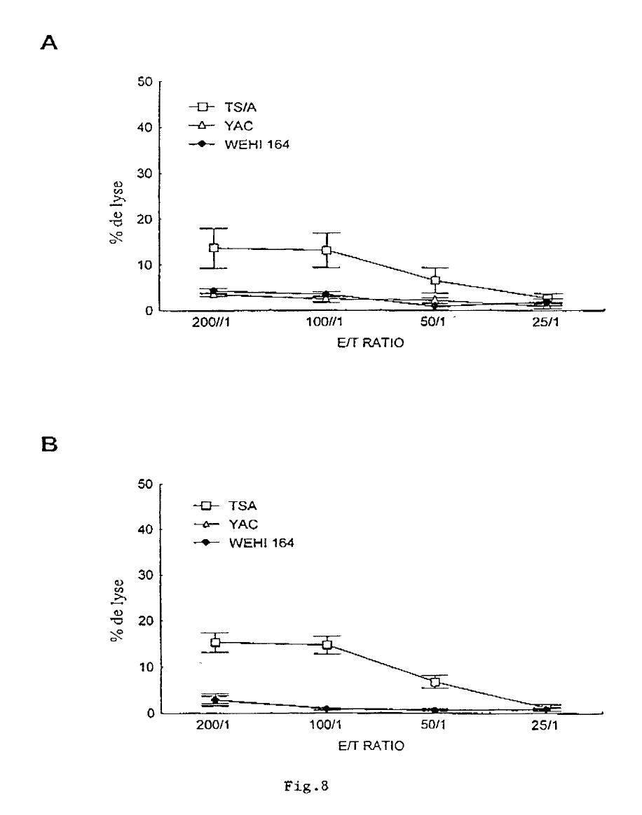

FIG. 8: CTL antitumor activity in mice having rejected the TS/A cells after administration of KY/IL-2. A. Mice having received a subcutaneous administration. B. Mice having received an intranasal administration.

Reference will be made in example 2 to the following figures:

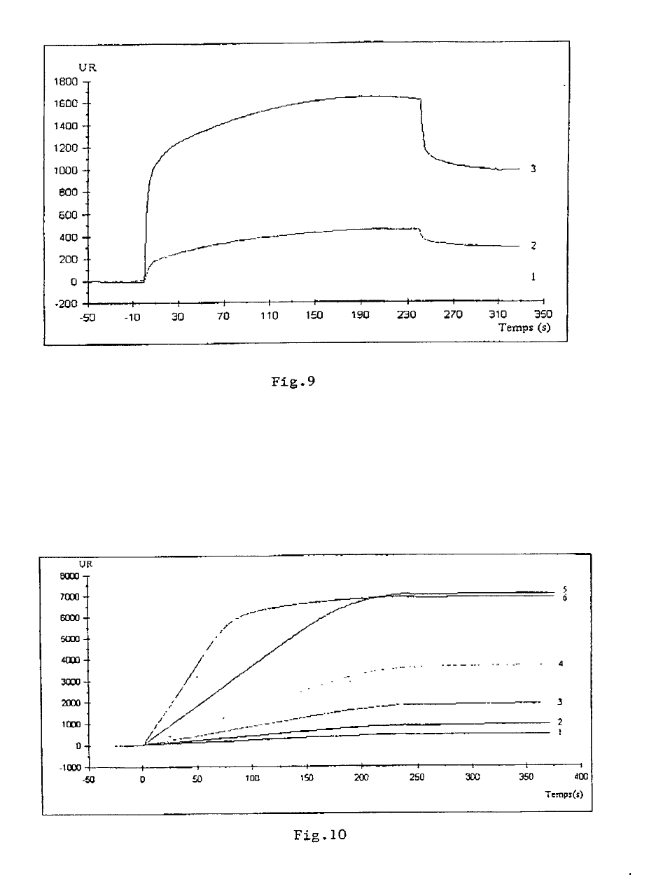

FIG. 9: Biacore analysis (surface plasmon resonance) of the combination of CTB with the BVSM™ as a function of the CTB concentration.

FIG. 10: Biacore analysis of the combination of MPL with the BVSM™ as a function of the MPL concentration.

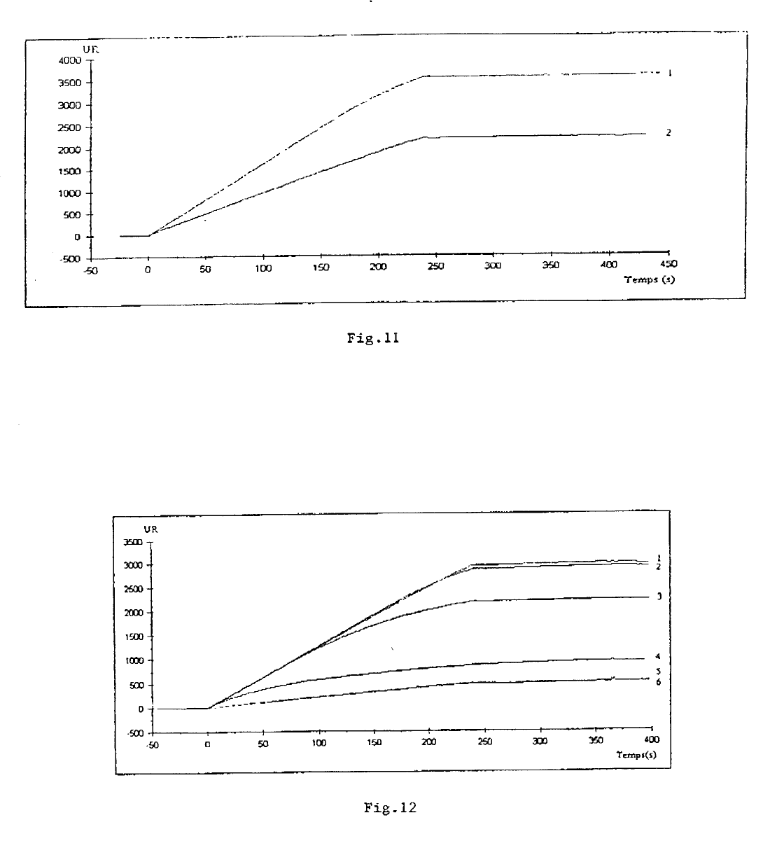

FIG. 11: Inhibition of the combination of the split B/Harbin vaccine on the BVSM in the presence of an increasing quantity of CTB.

FIG. 12: Inhibition of the combination of the split B/Harbin vaccine on the BVSM in the presence of an increasing quantity of MPL.

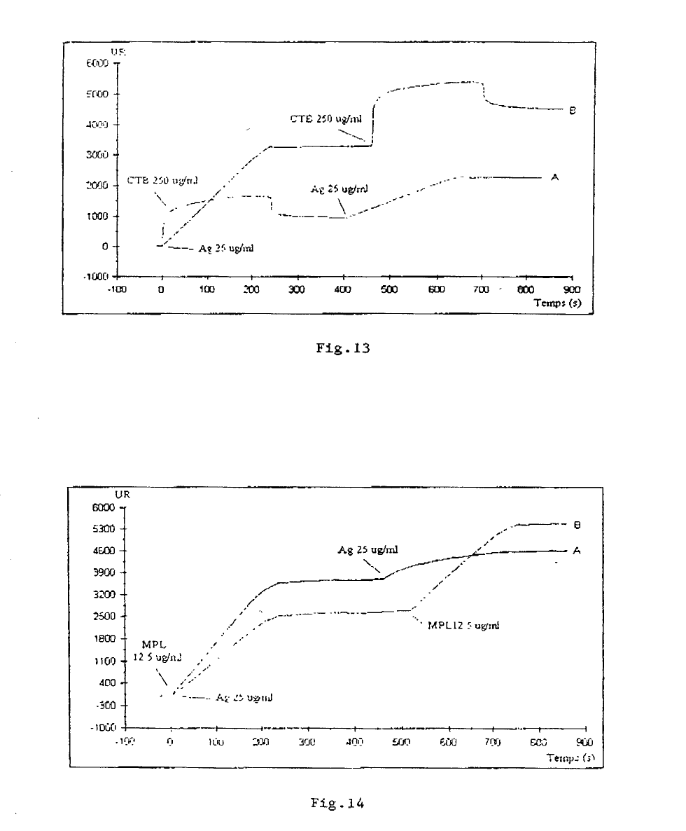

FIG. 13: Sensorgrams obtained by comparing two modes of preparation of CTB and flu antigens on the BVSM immobilized on the sensor chip HPA. In a first step, the CTB then the antigen were deposited successively on the BVSM (curve A) and inversely (curve B).

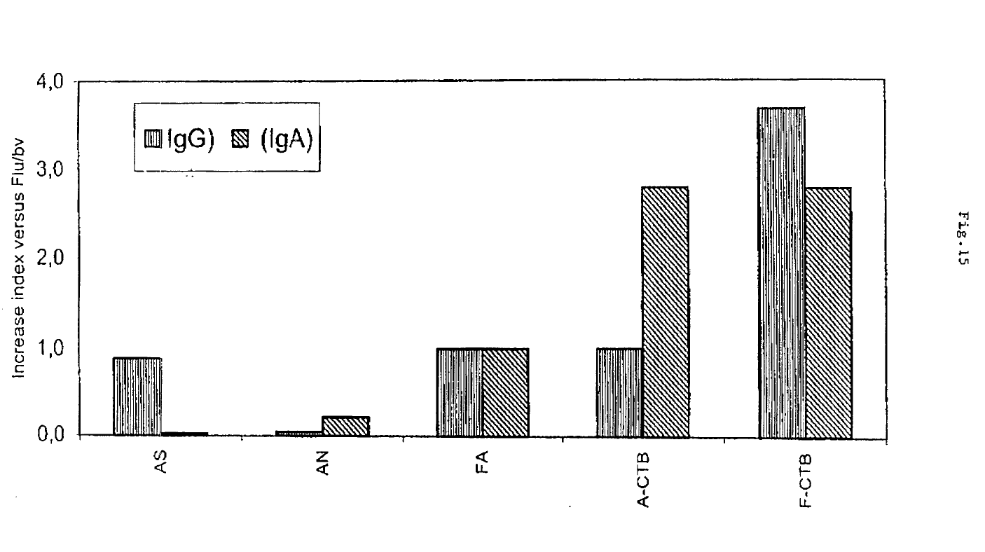

FIG. 14: Sensorgrams obtained by comparing two modes of preparation of MPL and flu antigens on the BVSM immobilized on the sensor chip HPA. In a first step, the MPL then the antigen were deposited successively on the BVSM (curve A) and inversely (curve B).

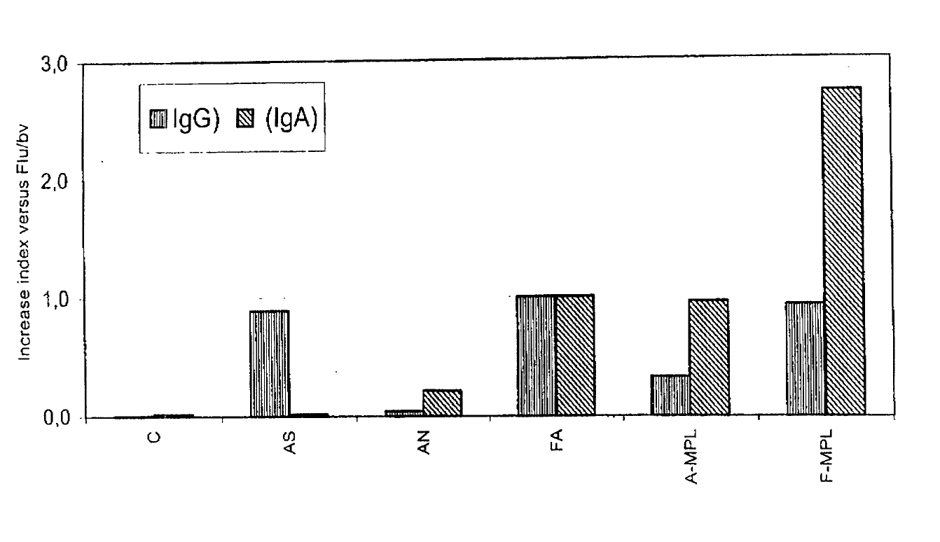

FIG. 15: specific titration of the split B/Harbin vaccine from serum pools (IgG) and nasal secretions (IgA) from mice to which had been administrated the trivalent formulations and the control antigens combined or not combined with CTB.

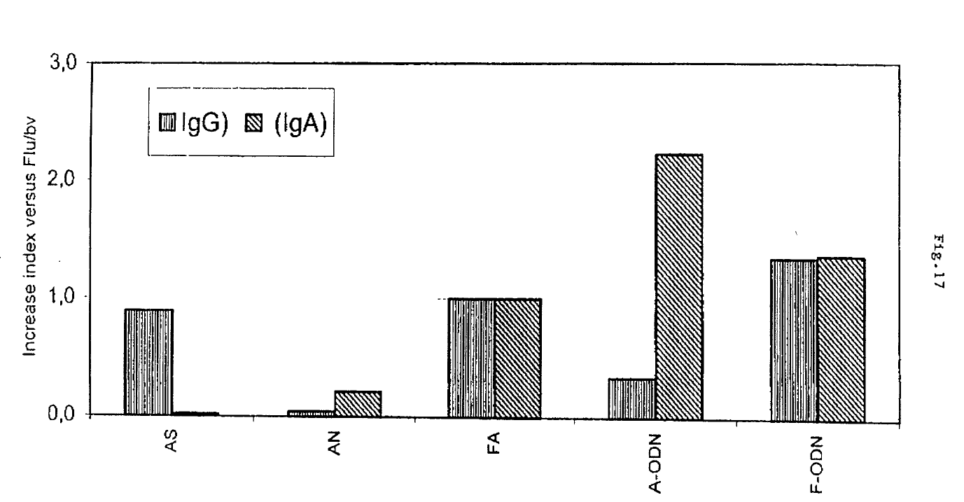

FIG. 16: specific titration of the split B/Harbin vaccine from serum pools (IgG) and nasal secretions (IgA) from mice to which had been administrated the trivalent formulations and the control antigens combined or not combined with MPL.

FIG. 17: represents the specific titration of the split B/Harbin vaccine from serum pools (IgG) and nasal secretions (IgA) from mice to which had been administrated the trivalent formulations and the control antigens combined or not combined with oligonucleotides.

### EXAMPLE 1

### Preparation and Biological Activity of BVSM-IL2

1) Characterization of the Particles Charged with IL-2

A) Preparation of Particles Charged with IL-2

The vectors used (BVSM) in this example have already been described: they comprise cationic vectors with a lipid layer of DPPC-cholesterol.

The interleukin-2 (IL-2) employed was obtained from the Chiron company in the form of lyophilized recombinant IL-2, prepared such that 18·10⁶ IU=1.1 mg of lyophilized protein.

A composition according to the invention was obtained by binding BVSM-IL2 by the simple mixing of the two components in a weight ratio of 10/1 in a buffered saline solution (PBS).

B. Determination of the Optimal BVSM-IL2 Ratio

The optimal mass ratio of BVSM in relation to the protein was determined by ELISA analysis according to a method well known to the expert in the field.

Briefly, the wells are covered with an anti-IL2 antibody and saturated with BSA (bovine serum albumin). The IL-2 preparation to be tested is added and then a second biotinylated anti-IL2 antibody is added. The addition of streptavidin bound to a peroxidase followed by the addition of a substrate of said peroxidase enables determination of the quantity of IL-2 present in the solution by colorimetry. The values obtained are expressed in Arbitrary Units, such that the value increases with increased levels of IL-2 in the tested solution.

The tested preparations were preparations in which the same quantity of IL-2 was introduced in the presence of vectors in various proportions or in their absence, with or without BSA as stabilizer of the proteins.

The results are shown in FIG. 1. It can be seen that approximately the same levels of available IL-2 were obtained in the preparations containing BSA, or a level of particle vectors such that the BVSM/IL-2 mass ratio was 10/1. When IL-2 was by itself in PBS or when the mass ratio was lower, there was less IL-2 available in the solution.

These results demonstrate that the vectors are as effective as albumin for stabilizing IL-2 and preventing a drop in the effective concentration.

C. Determination of the Combination Rate of IL-2 with the BVSM

The combination rate of IL-2 with the vectors was determined using the plasmon surface resonance technique in a Biacore X device according to the manufacturer's instructions. This method enables detection of the differences in the refraction index of a surface layer of a solution in contact with the detection chip by illumination with a monochromatic polarized light.

The operating protocol is as follows:

the BVSM (0.05 g/l) are adsorbed on the surface of the detection chip;

free IL-2 (1-6 mg/ml) is injected in order to determine the standard curve (expressed as resonance units, RU) which is a function of the concentration of injected protein;

after regeneration, we injected a preparation like that prepared in example 1.A in which were present the BVSM-IL2 as well as free IL-2. Only the free IL-2 can combine with the BVSM fixed on the surface of the detection chip.

This protocol enables determination of the concentration of free IL-2 in the formulation of example 1.A and makes it possible to deduce from it the BVSM-IL2 combination rate.

The data in FIG. 2 enable calculation of said combination rate which was 85%.

2) Analysis of the Biological Activity of IL-2 Combined with Different Vectors

These examples demonstrate the importance of the composition of the cores and layers of the vector on the biological activity of IL-2.

Four different types of vectors were used:

two vectors with anionic cores with a lipid layer containing

a. a Phospholipon/cholesterol mixture (P PLpon/Chol), or

b. a DPPC/cholesterol mixture (P DPPC/Chol, or PY),

two vectors with cationic cores and a membrane containing

c. a Phospholipon/cholesterol mixture (QAE PLpon/Chol), or

d. a DPPC/cholesterol mixture (QAE DPPC/Chol, or KY).

The IL-2 was added to the BVSM in a mass ratio of 1/10.

The biological activity of the IL-2 combined with the different BVSM was evaluated by measuring the proliferation of mononuclear blood cells, calculated by incorporation of thymidine tagged with H³. The cells were first stimulated by compounds (PHA) which induce expression of the CD25 receptor, which has a strong affinity for IL-2.

It can be seen (FIG. 3) that all of the BVSM made it possible to obtain an activity equivalent to that of unformulated control IL-2 in vitro, but that BVSM QAE DPPC/Chol even improved this biological activity.

3) Study of the Stability of the IL-2 Preparations

Preparations of IL-2 in solution are generally not stable at 4° C., such that the IL-2 loses its biological activity.

The BVSM-IL2 preparation (QAE/DPPC/Chol) remained stable after two months of storage at 4° C., with 95% of the biological activity of the fresh IL-2 being maintained as measured by incorporation of H³-tagged thymidine in murine CTLL-2 cells.

4) In Vivo Tests. TS/A Tumor

The activity of the BVSM-IL2 (KY/IL-2 mass ratio 10/1) was tested in the TS/A tumor model (undifferentiated, nonimmunogenic mammary adenocarcinoma), in a tumor implantation rejection model or a model of treatment of previously implanted tumors.

A. Rejection of Tumor Implantation

A.1. Co-Administration in the Same Flank

We co-administered 5·10⁴ tumor cells to female BALB/c mice via the subcutaneous route (s.c.) as well as the BVSM-IL2 (KY/IL-2) corresponding to the IL-2 concentration indicated in FIG. 4. As controls, we also administered the vectors alone or unformulated IL-2, or simple PBS. The implantation and evolution of the size of the tumors were measured.

FIG. 4 clearly shows that implantation of the tumor cells and growth of the tumor takes place after administration of the vectors alone or IL-2 alone, whereas IL-2 formulated with the KY vectors retards tumor growth.

A.2 Contralateral Administration

We administered 5·10⁴ TS/A tumor cells to BALB/c mice via the s.c. route as well as KY/IL-2 corresponding to the IL-2 concentrations indicated in FIG. 5 in the contralateral flank. We administered unformulated IL-2 or PBS as controls.

FIG. 5 shows that the contralateral administration of IL-2 complexed to the vectors diminishes tumor growth, a result not seen with unformulated IL-2 even when used at a 100-times higher dose.

B. Therapeutic and Protective Effects on an Implanted Tumor; Administration Via the Subcutaneous Route

B.1. Therapeutic Effect

Six days after s.c. administration of 5·10⁴ TS/A cells to 10 mice/group, we administered the KY/Il-2 via the s.c. route in the contralateral flank. The administration was performed at a single site (5·10³ units of IL-2) or at five distinct sites with the injected dose in this case being 1·10³ units per site.

In this type of model, the tumor is implanted and is palpable (surface of circa 40 mm). The augmentation of the size of the tumor is evaluated. FIG. 6.A shows that IL-2 complexed with the vectors slowed down tumor growth to a greater extent than IL-2 alone. Furthermore, it appears that injections of small doses at multiple sites is more effective than injection of a higher dose at a single site.

Three animals out of the 10 mice that received the KY/IL-2 at a single site did not present detectable tumors after 30 days, whereas 6 mice out of the 10 that received the KY/IL-2 at five administration sites were also without detectable tumors. These nine animals did not redevelop tumors later.

B.2. Protective Effect

We reinjected 25·10⁴ TS/A cells in the nine mice described above; they no longer presented tumors 46 days after administration of the IL-2. Naive mice were used as controls.

FIG. 6.B shows that the prior administration of KY/IL-2 partially protects the animals against a new challenge. In fact, four mice out of nine did not develop tumors and the tumoral development was slowed down in the other animals compared to the naive animals.

C. Therapeutic and Protective Effects on an Implanted Tumor; Administration Via the Intranasal Route

C.1. Therapeutic Effect

Commencing six days after s.c. administration of 5·10⁴ TS/A cells to 10 mice/group, we administered KY/IL-2 via the intranasal route for a period of 5 days. The administration was performed once or twice daily. The controls employed were IL-2 alone (administered twice daily), the vectors alone or PBS.

In this type of model, the tumor is implanted and is palpable (surface of circa 40 mm). The augmentation of the size of the tumor is evaluated. FIG. 6.A shows that IL-2 complexed with the vectors slowed down tumor growth to a greater extent than IL-2 alone. The number of administrations per day did not appear to make any difference in the results obtained.

Four animals out of the 10 mice that received the KY/IL-2 via the intranasal route once daily did not present detectable tumors after 35 days, whereas 6 mice out of the 10 that received the KY/IL-2 twice daily were also without detectable tumors. These ten animals did not redevelop tumors later.

C.2. Protective Effect

We reinjected 25·10⁴ TS/A cells in the ten mice described above; they no longer presented tumors 48 days after administration of the IL-2. Naive mice were used as controls.

FIG. 7.B shows that the prior administration of KY/IL-2 partially protects the animals against a new challenge. In fact, four mice out of ten did not develop tumors and the tumoral development was slowed down in the other animals compared to the naive animals.

5) Induction of CTL

We isolated the splenocytes from four mice treated via the subcutaneous route (after 75 days) or the intranasal route (after 79 days) that had rejected the implanted tumor and had not redeveloped tumors after a new challenge with a higher dose (sections 4.B and 4.C).

The splenocytes were stimulated in vitro with TS/A cells for 6 days and the CTL activity against the TS/A cells was assessed.

We found that the mice having received the KY/IL-2 via the subcutaneous route expressed a specific CTL activity of the TS/A (FIG. 8.A) because neither the syngenic WEHI 164 nor the YAC cells were lysed.

We also observed a specific CTL activity in the mice having received the KY/IL-2 via the intranasal route.

### EXAMPLE 2

### Effect in the Mouse of BVSM on the Immunogenicity of a Trivalent Split Influenza Vaccine with Different Adjuvants, and Effect in the Mouse of the Co-Administration of the Formulation and Different Adjuvants on the Immunogenicity of a Trivalent Split Influenza Vaccine

I—Material and Methods

1) Material

The animals employed were female BALB/cJ/Rj mice (aged 10 weeks at the beginning of the study) obtained from the Janvier breeding center (Route des Chênes-Sec —B.P. 5—Le Genest-Saint-Isle). They were acclimated for 7 days, housed 6 mice per cage, prior to commencement of the study.

2) Products

a) BVSM™

The BVSM™ used was of the type KY:QAE=2 mEq−DPPC/cholesterol. It corresponds to a polysaccharide core grafted by glycidyl trimethylammonium and enclosed in a layer of DPPC/cholesterol. This type of vector is described in EP 687 173.

b) Adjuvants

The following four adjuvants were used:

Subunit B of the cholera toxin (CTB) (ref. C9903, Sigma, St Quentin Fallavier, France).

Monophosphoryl, lipid A (MPL) (ref. R-350, Ribi Immunochem Research, Inc., Hamilton, Mo.).

Recombinant IL-2, Proleukin, activity 16.3·10⁶ U/mg (Chiron France, Suresnes, France).

Oligonucleotides ODN: 5TCCATGACGTTCCTGAC′ (Eurogentec, Brussels, Belgium).

c) Trivalent Split Influenza Vaccine

A solution at 250 μg of HA/ml (83.3 μg/strain) of trivalent split influenza vaccine was prepared with 3 monovalent split-virus egg vaccines from Biochem Pharma (Canada).

the monovalent split-virus egg vaccine produced from strain B/Harbin 7/94.

the monovalent split-virus egg vaccine produced from strain A/Johannesburg 82/96.

the monovalent split-virus egg vaccine produced from strain A/Nanchang 933/95.

These split vaccines were constituted by a mixture of viral proteins, notably hemagglutinin (HA) and neuraminidase.

d) Formulation

Antigen Formulation+BVSM™

A formulation at 250 μg of HA/ml (83.3 μg/strain) of trivalent split influenza vaccine was prepared with the three abovementioned split influenza vaccines so as to obtain an HA/BVSM ratio equal to 1/89: 250 μg of trivalent HA/22.25 mg of BVSM/ml.

This formulation was obtained by mixing at equal volume the 3 formulas at 250 μg of HA/22.25 mg of BVSMT/ml of each of the monovalent split vaccines.

Antigen Formulation+BVSM™+Adjuvant

Four other formulations with adjuvant were prepared from the antigen formulation+BVSM™. These formulations were obtained by dilutions of the trivalent formulation at 250 μg of HA/ml in the presence and by addition of adjuvant. Dilution enabled adjustment of the dose of monovalent HA/mouse/mouse/immunization to 1.2 μg, i.e., 60 μg of monovalent HA/ml and of adding the adjuvant to the volume used for dilution.

e) Controls

Three types of controls were prepared:

trivalent split vaccine control, referred to below as AS and AN, prepared by mixing at equal volume the 3 solutions at 250 μg of HA/ml of each of the monovalent split vaccines;

trivalent split vaccine controls+adjuvant;

a naive control (PBS 0.22×).

The trivalent split vaccine solution was prepared in phosphate buffered saline or in sterile water for subcutaneous (AS) or intranasal (AN) administration respectively.

3) Methods

The different formulations (adjuvant±BVSM±Ag) were administered on days 0 and 21 according to the following diagram:

| | | |
|---|---|---|
| | PBS control | i.n. |
| C: | Naive control | i.n. |
| AS: | Ag | s.c. |
| AN: | Ag | i.n. |
| FA: | Ag + BVSM | i.n. |
| Ax: | Ag + Adj X | i.n. |
| FX: | Ag + BVSM + Adj X | i.n. |

The immunogenicity was evaluated after the booster on day 35:

at the serum level, detection by ELISA of the specific IgG of each monovalent split vaccine,

at the mucus level, detection by ELISA in the nasal and/or vaginal secretions of specific IgA of each monovalent split vaccine.

a) Immunizations of the Mice

Administrations

Administration of the formulations (BVSM±Adj±Ag) was performed without anesthesia either:

via the subcutaneous route: 100 μl with a 1-ml syringe at the dorsal level for the controls,

via the intranasal route: 20 μl (10 μl/nostril) with a 10 μl-micropipette for the test samples and the controls.

The immunization comprised two administrations on day 0 and day 21 performed according to the same modality for each group.

Collection of Blood Samples

An approximately 0.3 ml blood sample was collected from the retro-orbital vein on day 0 (control) and day 35.

After formation of a clot and centrifugation, the serum was frozen at −20° C. until use.

Collection of Nasal Secretions

The nasal cavities were washed with 500 μl of phosphate saline buffer −1% BSA introduced in the trachea in the direction of the nasal concha. The operation was repeated 3 times with the same PBS −1% BSA. The nasal specimen was stored in an Eppendorf tube at −20° C. Nasal secretions were collected on day 35.

b) Analysis of the Samples

The analysis of the serums and nasal secretions was performed by ELISA. Briefly, the microplates (Nunc, Maxisorb Immunoplate, Polylabo) were coated with flu vaccine (100 ng of HA/well in a carbonate buffer, pH 9.6, 2 h at 37° C.). After rinsing (three times with PBS buffer-Tween, pH 7.6) then saturation by 250 μl/well of a 3% PBS-BSA solution (1 h at 37° C.), the sets of serums or nasal secretions were incubated for 1 hour at 37° C., and rinsed again with a PBS-Tween solution, pH 7.6. After rinsing (three times), the antibodies were detected by murine anti-IgG (ref. A4416 Sigma) or murine anti-IgA (ref. A4789 sigma) coupled with peroxidase. After a final rinsing step (five times), the OPD substrate was added (ref. P8287, Sigma 1 pastille in 5 ml of development buffer+50 μl of H₂O₂, 100 μl/well). After 30 minutes of incubation at 37° C., 1N hydrochloric acid (ref. 30024-290, Prolabo) was added (50 μl/well) and the absorbance was measured at 490 nm. The titers were defined as the inverse of the dilution that made it possible to obtain an OD value of 0.1.

II—Influence of the Mode of Preparation of the BVSM™/Adjuvant/Influenza Formulations

In order to evaluate the best mode of preparation of the BVSM/adjuvant/influenza formulations, the combination of influenza split vaccine on the BVSM was studied in the presence of adjuvant (CTB or MPL) using Biacore X surface plasmon resonance (SPR) (Pharmacia Biosensor Inc.). In this study, 20 μl of a suspension of BVSM (50 μg/ml in PBS 0.3×) was introduced on hydrophobic sensor chips (HPA, Biacore, BR-1000-30) at 5 μl/min.

FIG. 9 shows the sensorgram obtained by combination of CTB and the BVSM immobilized on the sensor chip HPA as a function of the CTB concentration (1) 2.5 μg/ml; 2) 25 μg/ml; 3) 250 μg/ml in 45 mM PBS) (which corresponds to 0.3×).

FIG. 10 shows the sensorgram obtained by combination of MPL and the BVSM immobilized on the sensor chip HPA as a function of the MPL concentration (1) 1.56 μg/ml; 2) 3.125 μg/ml; 3) 6.25 μg/ml; 4) 12.5 μg/ml; 5) 25 μg/ml; 6) 50 μg/ml in 45 mM PBS).

In both the case of CTB and that of MPL, the sensorgrams obtained confirmed the combination capacity of the BVSM and the adjuvants.

The mode of preparation of the BVSM/adjuvant/split influenza vaccine formulations was then studied. Two methods were used.

In the first method, a solution of adjuvant was introduced after addition of the split influenza vaccine.

In the second method, the split influenza vaccine was introduced prior to the addition of the adjuvant.

FIG. 11 summarizes the results obtained in the case of a split B/Harbin vaccine. Variable quantities of CTB were introduced on the immobilized BVSM (1) no CTB, 2) 2.5 μg/ml, 3) 25 μg/ml, 4) 250 μg/ml in 45 mM PBS). The monovalent split B/Harbin vaccine at 25 μg/ml was then deposited on the BVSM carrying adjuvant. There is clearly visible an inhibition of the combination of the split vaccine on the BVSM when the CTB is added before the split vaccine.

Similarly, FIG. 12 summarizes the results obtained in the case of MPL and a split B/Hardin vaccine. Variable quantities of MPL were introduced on the immobilized BVSM (1) 1.56 μg/ml; 2) 3.12 μg/ml; 3) 6.25 mg/ml; 4) 12.5 μg/ml; 5) 25 μg/ml; 6) 50 μg/ml in 45 mM PBS). The split vaccine at 25 μg/ml was then deposited on the BVSM carrying adjuvants. As was the case for CTB and proportionally to the quantity of MPL, there was an inhibition of the combination of the split vaccine with the BVSM when the adjuvant was added before the split vaccine.

FIG. 13 represents the sensorgrams obtaining by comparing the two modes of preparation in the case of CTB. In A, the CTB was added onto the immobilized BVSM and then the monovalent split vaccine was introduced.

In B, the monovalent split vaccine was added onto the immobilized BVSM and then the CTB was introduced.

It can be seen that when the antigen was added before the CTB, there was a decrease in this combination of the split vaccine with the BVSM. Inversely, when the CTB was added after the split vaccine, the BVSM enabled combination of an additional quantity of material corresponding to the CTB. Thus, in order to preserve the role of BVSM as antigen carrier, it is important to maintain a mode of preparation in which the CTB is added to the BVSM/split vaccine formulations.

Similarly, FIG. 14 represents the sensorgrams obtained by comparing the two modes of preparation in the case of MPL.

In A, the MPL was added before the split vaccine. It was the inverse in B.

When the antigen was added before the MPL, there was no significant modification of the split vaccine/BVSM combination but the BVSM was able to combine an additional quantity of material corresponding to MPL.

Thus it seems possible to define a mode of preparation of adjuvant-carrying formulations in which:

the adjuvant is added onto the BVSM/antigen formulations,

the role of BVSM as antigen carrier is preserved.

III—Effect of CTB on the Immunologic Response of the BVSM/Influenza Formulations

1) Protocol

The antigen+BVSM™ formulation was prepared as stated in the “Material and methods” section.

A control was prepared under the same conditions but in the absence of BVSM™, resulting in a trivalent split vaccine at 250 μg of HA (83 μg/strain)/ml.

A solution of CTB was added on each of these preparations in order to obtain the corresponding adjuvant-carrying formulations and controls.

Thirty-six female BALB/cJ/Rj mice (aged 10 weeks at the beginning of the study) were divided into 6 groups at the rate of 6 mice per group. The treatment was performed as previously described for each group. Table 1 below summarizes the treatment of each group.

TABLE 1

| | Group | | Formulation administered |
|---|---|---|---|
| Code | (number of mice) | Route | Quantity/dose |
| C | Naive control | i.n. | 20 μl PBS 80 mOsm/kg |
| FA | Ag + BVSM | i.n. | 3.6 μg HA + 320.4 μg BVSM |
| AS | Ag s.c. | s.c. | 3.6 μg HA |
| AN | Ag i.n. | i.n. | 3.6 μg HA |
| F^{CTB} | Ag + BVSM + CTB | i.n. | 3.6 μg HA + 320.44 μg BVSM + |
| | | | 1 μg CTB |
| A^{CTB} | Ag + CTB | i.n. | 3.6 μg HA + 1 μg CTB |

Blood and nasal secretion samples were collected for each group and were analyzed as specified in the “Material and methods” section.

2) Results

FIG. 15 summarizes the results obtained in specific titration of the split B/Hardin vaccine from pools of mouse serums (IgG) and nasal secretions (IgA). It makes it possible to compare the results of the trivalent formulations containing CTB (F-CTB) with various controls. These controls are either control antigens alone, or control antigens combined with CTB, or the reference formulation (HA/BVSM ratio: 1/89). These results were confirmed by analysis of the individual response against the split B/Harbin and A/Nanchang vaccines.

Unexpectedly, the reference formulation (FA) induced a level of type G antibodies equivalent to the control antigen alone administered via the subcutaneous route (AS) and a higher level of IgG than the control alone administered via the nasal route (AN) (22.4×).

The controls comprising antigens combined with CTB (ACTB) administered via the nasal route induced markedly higher levels of IgG than the free antigen administered via the same route (augmentation of 22.4×). In parallel these formulations (Ag+CTB) (ACTB) induced a strong mucosal immunity.

The formulation combined with the adjuvant CTB (FCTB) induced a specific IgG response markedly higher than the reference formulation (FA) and its reference control (ACTB). There exists for this adjuvant a noteworthy synergy effect between CTB and the BVSM™; thus the serum IgG levels obtained were multiplied by a factor of 3.7 in relation to the reference formulation FA. Inversely and in the presence of a noteworthy mucosal adjuvant activity of CTB, with certainly an effect of saturation of the biological response, it is difficult at this stage to define the synergy potential of the IgA response for this type of adjuvant and the BVSM™.

IV—Effect of MPL on the Immunologic Response of the BVSM™/Influenza Formulations

1) Protocol

A trivalent formulation and the corresponding control were prepared at 250 μg of HA/ml (83.3 μg/strain)/of split influenza vaccine.

A solution of MPL was added onto each of these preparations so as to obtain the corresponding adjuvant-carrying formulations and controls.

Thirty-six female BALB/cJ/Rj mice (aged 10 weeks at the beginning of the study) were divided into 6 groups at the rate of 6 mice per group. The treatment was performed as previously described in the “Material and methods” section. Table 2 below summarizes the treatment of each group.

TABLE 2

| | Group | | Formulation administered |
|---|---|---|---|
| Code | (number of mice) | Route | Quantity/dose |
| C | Naive control (6) | i.n. | 20 μl PBS 80 mOsm/kg |
| FA | Ag + BVSM | i.n. | 3.6 μg HA + 320.4 μg BVSM |
| AS | Ag s.c. | s.c. | 3.6 μg HA |
| AN | Ag i.n. | i.n. | 3.6 μg HA |
| FMPL | Ag + BVSM + MPL | i.n. | 3.6 μg HA + 320.4 μg BVSM + |
| | | | 5 μg MPL |
| AMPL | Ag + MPL | i.n. | 3 μg HA + 5 μg MPL |

Blood and nasal secretion samples were collected for each group as described in the method part.

Analysis of the serums and nasal secretions was performed by ELISA.

2) Results

FIG. 16 summarizes the results obtained in specific titration of the split B/Hardin vaccine from pools of serums (IgG) and nasal secretions (IgA) from mice to which had been administered formulations carrying the adjuvant MPL. These results were confirmed by analysis of the individual response against the split B/Harbin and A/Nanchang vaccines.

The split influenza virus vaccines combined with BVSM™ or MPL administered via the nasal route induced a serum IgG response markedly higher than the free antigen administered by the same route (respective augmentations of 22.4× and 7.3×). In parallel these formulations induced a strong mucosal immunity.

The formulation combined with the adjuvant MPL induced a specific IgG response similar to the reference formulation. Inversely for this adjuvant there exists a noteworthy synergy effect between MPL and the BVSM™. Thus the levels of IgA obtained were multiplied by a factor of 2.7 in relation to the (Ag/BVSM) reference formulation (FA). Thus, to the inverse of the IgG response, a strong synergy potential could be demonstrated between MPL and the BVSM™ for the mucosal response.

V—Effect of Oligonucleotides on the Immunologic Response of the BVSM/Influenza Formulations

1) Protocol

A trivalent formulation and the corresponding control were prepared at 250 μg of HA/ml (83.3 μg/strain)/of split influenza vaccine.

A solution of oligonucleotides (ODN) was added onto each of these preparations so as to obtain the corresponding adjuvant-carrying formulations and controls.

Forty-two female BALB/cJ/Rj mice (aged 10 weeks at the beginning of the study) were divided into 7 groups at the rate of 6 mice per group. The treatment was performed for each group as described in the “Material and methods” section. Table 3 below summarizes the treatment of each group.

TABLE 3

| | Group | | Formulation administered |
|---|---|---|---|
| Code | (number of mice) | Route | Quantity/dose |
| C | Naive control | i.n. | 20 μl PBS 80 mOsm/kg |
| FA | Ag + BVSM | i.n. | 3.6 μg HA + 320.4 μg BVSM |
| AS | Ag s.c. | s.c. | 3.6 μg HA |
| AN | Ag i.n. | i.n. | 3.6 μg HA |
| F_{ODN} | Ag + BVSM + ODN | i.n. | 3.6 μg HA + 320.4 μg BVSM + |
| | | | 1 μg ODN1 |
| A_{ODN} | Ag + ODN | i.n. | 3 μg HA + 1 μg ODN1 |

Blood and nasal secretion samples were collected for each group as described in the method part.

Analysis of the serums and nasal secretions was performed by ELISA as described above.

2) Results

FIG. 17 summarizes the results obtained in specific titration of the split B/Hardin vaccine from pools of serums (IgG) and nasal secretions (IgA) from mice to which had been administered formulations carrying the adjuvant oligonucleotides. These results were confirmed by analysis of the individual response against the split B/Harbin and A/Nanchang vaccines.

The split influenza virus vaccine combined with BVSM™ or with ODN administered by the nasal route induced a clearly higher serum IgG response than the free antigen administered by the same route (respective augmentations of 22.4× and 7.3×). In parallel these formulations induced a strong mucosal immunity.

The formulation combined with the adjuvant ODN induced a specific IgG response superior to that of the reference formulation and to that of its reference control (Ag+ODN). For this adjuvant there appears to exist an additive effect of the responses, the IgG levels obtained for the formulation being equal to the sum of the responses obtained for the antigen+ODN1(AODN1) and for the reference formulation (Ag+BVSM™) (FA).

VI—Modification of the IgG2a/Igg1 Balance by Addition of Adjuvants to the BVSM/Influenza Formulations

1) Protocol

A trivalent formulation and the corresponding control were prepared at 250 μg of HA/ml (83.3 μg/strain)/of split influenza vaccine. Three adjuvant-containing formulations were obtained from this trivalent formulation:

BVSM™/Ag/CTB: by addition on the trivalent formulation of a solution of CTB.

BVSM™/Ag/IL2: by addition on the trivalent formulation of a solution of recombinant IL-2.

BVSM™/Ag/MPL: by addition on the trivalent formulation of a solution of MPL.

Fifty-four female BALB/cJ/Rj mice (aged 10 weeks at the beginning of the study) were divided into 9 groups at the rate of 6 mice per group. The treatment was performed for each group as described in the “Material and methods” section.

Table 4 below summarizes these treatments.

TABLE 4

| | Group | | Formulation administered |
|---|---|---|---|
| Code | (number of mice) | Route | Quantity/dose |
| C | Naive control | i.n. | 20 μl PBS 80 mOsm/kg |
| FA | Ag + BVSM | i.n. | 3.6 μg HA + 320.4 μg BVSM |
| AS | Ag s.c. | s.c. | 3.6 μg HA |
| F_{CTB} | Ag + BVSM + CTB | i.n. | 3.6 μg HA + 320.4 μg BVSM + |
| | | | 1 μg CTB |
| A_{CTB} | Ag + CTB | i.n. | 3.6 μg HA + 1 μg CTB |
| F_{MPL} | Ag + BVSM + MPL | i.n. | 3 μg HA + 320.4 μg BVSM + |
| | | | 5 μg MPL |
| A_{MPL} | Ag + MPL (6) | i.n. | 3.6 μg HA + 5 μg MPL |
| F_{IL} | Ag + BVSM + IL-2 | i.n. | 3 μg HA + 320.4 μg BVSM + |
| | | | 0.613 μg IL-2 |
| A_{IL} | Ag + IL-2 | i.n. | 3.6 μg HA + 0.613 μg IL-2 |

Analysis of the serums was performed by ELISA as described above using either a murine anti-IgG2a or a murine anti-IgG1.

2) Results

Table 5 below summarizes the results obtained in terms of gamma-globulin subtype (IgG2a and IgG1) from the different adjuvant-containing BVSM™/influenza formulations and the corresponding controls after nasal administration. In comparison with the free antigen administered by the subcutaneous route, the BVSM™/influenza formulation administered by the nasal route induced a modest augmentation of specific IgG2a production.

By comparison, the addition of CTB to the Ag/BVSM™ formulation induced a clear augmentation of the IgG2a production (multiplication by 5.4 of the IgG2a/IgG1 index versus Ag s.c.). It is important to note that this modification of the Th1/Th2 balance was not predictable from the results obtained by the combination of CTB with the antigens. In fact, in this control group there was a reduction in the IgG2a/IgG1 index.

TABLE 5

| | | | | | Ratio |
|---|---|---|---|---|---|
| | | | | IgG2a/IgG1 | versus |
| | Route | IgG2a | IgG1 | index | Ag s.c. |
| Naive control | i.n. | 0 | 0 | 0 | 0.0 |
| Ag s.c. | s.c. | 157 | 23,355 | 6.7 | 1.0 |
| Ag i.n. | i.n. | 0 | 9793 | 0.0 | 0.0 |
| Ag + BVSM | i.n. | 572 | 65,699 | 8.7 | 1.3 |
| Ag + CTB | i.n. | 87 | 44,771 | 1.9 | 0.3 |
| Ag + BVSM + CTB | i.n. | 2506 | 68,877 | 36.4 | 5.4 |
| Ag + MPL | i.n. | 143 | 3003 | 47.6 | 7.1 |
| Ag + BVSM + MPL | i.n. | 101 | 10,561 | 9.6 | 1.4 |
| Ag + IL-2 | i.n. | 0 | 3234 | 0.0 | 0.0 |
| Ag + BVSM + IL-2 | i.n. | 26 | 24,952 | 1.0 | 0.2 |

Inversely, when MPL was combined with the split influenza vaccine it promoted the production of IgG2a (index of 47.6 versus 6.7 for the Ag s.c.) but only caused a minor modification of the Th1/Th2 balance after combination with the Ag/BVSM™ formulations.

Finally, the combination of IL-2 with the Ag/BVSM™ formulations enabled modification of the Th1/Th2 balance as we could see in the reduction of the IgG2a/IgG1 index.

Thus, even in the absence of quantitative modification of the immunologic response, the combination of adjuvants with the antigen/BVSM™ formulations enables induction of a qualitative modification of the immunologic response. This combination should enable a correct choice of the adjuvant used to adapt the Th1/Th2 balance to the proposed vaccine strategy.

1. Use of a vector of the type comprising a nonliquid hydrophilic core for the preparation of a medication intended for the treatment of cancers and/or viral diseases, said vector being combined in the medication with at least one substance other than an antigen capable of modulating the immune response.

2. Use according to claim 1, characterized in that the vector is of the type comprising a nonliquid hydrophilic core and an external layer constituted at least in part of amphiphilic compounds associated with the core by hydrophobic interactions and/or ionic bonds.

3. Use according to either claim 1 or 2, characterized in that the nonliquid hydrophilic core is constituted by a matrix of naturally or chemically cross-linked polysaccharides or oligosaccharides on which are grafted ionic ligands.

4. Use according to claim 3, characterized in that the ionic ligands have a positive charge.

5. Use according to claim 4, characterized in that the ionic ligands with a positive charge are quaternary ammoniums.

6. Use according to one of claims 2 to 5, characterized in that the external layer is formed by dipalmitoyl phosphatidyl choline (DPPC) and cholesterol.

7. Use according to claim 6, characterized in that the DPPC/cholesterol mass ratio is 70/30.

8. Use according to any one of the preceding claims, characterized in that the substance/vector weight ratio is comprised between circa 1% and 20%, preferably between circa 5% and 10%.

9. Use according to any one of the preceding claims, characterized in that the substance other than an antigen capable of modulating the immune response is a protein of therapeutic value which plays a role in the functioning of the immune system, an adjuvant or a substance capable of modifying the Th1/Th2 balance, or a mixture of these.

10. Use according to claim 9, characterized in that the protein of therapeutic value is a cytokine or a chemokine, preferably selected from among the group comprising the interleukins, the interferons, the TNF, the TGF, G-CSF, CM-CSF, MIF, RANTES or a mixture of these.

11. Use according to claim 10, characterized in that the protein of therapeutic value is interleukin-2.

12. Use according to claim 9, characterized in that the adjuvant is selected from the group comprising bacterial endotoxins, derivatives of liposaccharides, oligonucleotides, derivatives of saponin, ammonium salts and their derivatives, type DT or TT proteins, or a mixture of these.

13. Use of a vector according to any one of the preceding claims, characterized in that the cancer to be treated is of the nonimmunogenic or weakly immunogenic type.

14. Use of a vector according to any one of the preceding claims, characterized in the medication is intended for administration via the nasal or oral route.

15. Use of a vector comprising:
a) a nonliquid hydrophilic core constituted by a naturally or chemically cross-linked matrix of polysaccharides or oligosaccharides on which are grafted ionic ligands of positive or negative charge,
b) an external layer constituted at least in part by amphiphilic compounds associated in the core by hydrophobic interactions and/or ionic bonds, and
c) incorporating IL-2
for the preparation of a medication intended for the administration of IL-2 in injectable form in the absence of albumin.

16. Process for improving the immunomodulatory properties of a substance other than an antigen capable of modulating the immune response, characterized in that it comprises mixing of said substance with vectors of the type comprising a nonliquid hydrophilic core.

17. Process according to claim 16, characterized in that the vectors are of the type comprising a nonliquid hydrophilic core and an external layer constituted at least in part of amphiphilic compounds associated with the core by hydrophobic interactions and/or ionic bonds.

18. Process according to either claim 16 or 17, characterized in that the nonliquid hydrophilic core is constituted by a matrix of naturally or chemically cross-linked polysaccharides or oligosaccharides on which are grafted ionic ligands.

19. Process according to claim 18, characterized in that the ionic ligands have a positive charge.

20. Process according to claim 19, characterized in that the ionic ligands with a positive charge are quaternary ammoniums.

21. Process according to any one of claims 17 to 20, characterized in that the external layer is formed by dipalmitoyl phosphatidyl choline (DPPC) and cholesterol.

22. Process according to claim 21, characterized in that the DPPC/cholesterol mass ratio is 70/30.

23. Process according to any one of claims 16 to 22, characterized in that the substance/vector weight ratio is comprised between circa 1% and 20%, preferably between circa 5% and 10%.

24. Process according to any one of claims 16 to 23, characterized in that the substance other than an antigen capable of modulating the immune response is a protein of therapeutic value which plays a role in the functioning of the immune system.

25. Process according to claim 24, characterized in that the protein of therapeutic value is a cytokine or a chemokine, preferably selected from among the group comprising the interleukins, the interferons, the TNF, the TGF, G-CSF, CM-CSF, MIF, RANTES or a mixture of these.

26. Process according to any one of claims 16 to 23, characterized in that the substance other than an antigen capable of modulating the immune response is an adjuvant.

27. Process according to claim 26, characterized in that the adjuvant is selected from the group comprising bacterial endotoxins, derivatives of liposaccharides, oligonucleotides, derivatives of saponin, ammonium salts and their derivatives, type DT or TT proteins, or a mixture of these.

28. Process according to any one of claims 16 to 23, characterized in that the substance other than an antigen capable of modulating the immune response is a substance capable of modifying the Th1/Th2 balance.

29. Process according to any one of claims 16 to 23, characterized in that the substance other than an antigen capable of modulating the immune response is an immunosuppressant.

30. Pharmaceutical composition, characterized in that it comprises:
a vector of the type comprising a nonliquid hydrophilic core constituted by a matrix of naturally or chemically cross-linked polysaccharides or oligosaccharides on which are grafted ionic ligands and possibly an external layer constituted at least in part of amphiphilic compounds associated with the core by hydrophobic interactions and/or ionic bonds, and
at least one protein of therapeutic value, and/or an adjuvant and/or a substance capable of modifying the Th1/Th2 balance.

31. Pharmaceutical composition according to claim 30, characterized in that the external layer is formed by dipalmitoyl phosphatidyl choline (DPPC) and cholesterol.

32. Pharmaceutical composition according to either claim 30 or 31, characterized in that the substance/vector weight ratio is comprised between circa 1% and 20%, preferably between circa 5% and 10%.

33. Pharmaceutical composition according to one of claims 30 to 32, characterized in that the protein of therapeutic value is a cytokine or a chemokine, preferably selected from among the group comprising the interleukins, the interferons, the TNF, the TGF, G-CSF, CM-CSF, MIF, RANTES or a mixture of these.

34. Pharmaceutical composition according to claim 33, characterized in that the protein of therapeutic value is interleukin-2.

35. Pharmaceutical composition according to one of claims 30 to 32, characterized in that the adjuvant is selected from the group comprising bacterial endotoxins, derivatives of liposaccharides, oligonucleotides, derivatives of saponin, ammonium salts and their derivatives, type DT or TT proteins, or a mixture of these.

36. Use of a vector of the type comprising a nonliquid hydrophilic core for the preparation of a vaccine composition, said vector being mixed in the composition with at least one antigen and at least one substance capable of modulating the immunologic response to said antigen.

37. Use according to claim 36, characterized in that the vector is of the type comprising a nonliquid hydrophilic core and an external layer constituted at least in part of amphiphilic compounds associated with the core by hydrophobic interactions and/or ionic bonds.

38. Use according to either claim 36 or 37, characterized in that the nonliquid hydrophilic core is constituted by a matrix of naturally or chemically cross-linked polysaccharides or oligosaccharides on which are grafted ionic ligands.

39. Use according to claim 38, characterized in that the ionic ligands have a positive charge.

40. Use according to claim 39, characterized in that the ionic ligands with a positive charge are quaternary ammoniums.

41. Use according to one of claims 37 to 40, characterized in that the external layer is formed by dipalmitoyl phosphatidyl choline (DPPC) and cholesterol.

42. Use according to any one of claims 36 to 41, characterized in that the substance/vector weight ratio is comprised between circa 1% and 20%, preferably between circa 5% and 10%.

43. Use according to any one of claims 36 to 42, characterized in that the substance capable of modulating the immune response of the antigen is an adjuvant and/or a protein of therapeutic value and/or a substance capable of modifying the Th1/Th2 balance.

44. Use according to claim 43, characterized in that the adjuvant is selected from among the bacterial endotoxins, the derivatives of saponin, ammonium salts and their derivatives, type DT or TT proteins, the cytokines, the oligonucleotides or a mixture of these.

45. Use according to claim 43, characterized in that the protein of therapeutic value is a cytokine or a chemokine, preferably selected from among the group comprising the interleukins, the interferons, the TNF, the TGF, G-CSF, CM-CSF, MIF, RANTES or a mixture of these.

46. Use according to any one of claims 36 to 45, for the preparation of a vaccine composition intended for the treatment and/or prevention of viral diseases, notably AIDS and chronic hepatitis, or cancers.

47. Vaccine composition, characterized in that it comprises:
an antigen or a mixture of antigens,
a vector of the type comprising a nonliquid hydrophilic core constituted by a matrix of naturally or chemically cross-linked polysaccharides or oligosaccharides on which are grafted ionic ligands and possibly an external layer constituted at least in part of amphiphilic compounds associated with the core by hydrophobic interactions and/or ionic bonds, and
at least one substance capable of modulating the immunologic response to said antigen.

48. Vaccine composition according to claim 47, characterized in that the external layer is formed by dipalmitoyl phosphatidyl choline (DPPC) and cholesterol.

49. Vaccine composition according to either claim 47 or 48, characterized in that the substance capable of modulating the immunologic response of the antigen is an adjuvant and/or a protein of therapeutic value and/or a substance capable of modifying the Th1/Th2 balance.

50. Vaccine composition according to claim 49, characterized in that the adjuvant is selected from among bacterial endotoxins, derivatives of saponin, ammonium salts and their derivatives, type DT or TT proteins, cytokines, oligonucleotides or a mixture of these.

51. Vaccine composition according to claim 49, characterized in that the protein of therapeutic value is a cytokine or a chemokine, preferably selected from among the group comprising the interleukins, the interferons, the TNF, the TGF, G-CSF, CM-CSF, MIF, RANTES or a mixture of these.
